(19) United States
(12) Patent Application Publication
(10) Pub. No.: US 2026/0137445 A1
(43) Pub. Date: May 21, 2026

(54) BIPOLAR MEDICAL DEVICE

(71) Applicant: Boston Scientific Scimed, Inc., Maple Grove, MN (US)

(72) Inventors: Juan Pablo Ortiz Garcia, Heredia (CR); Gonzalo Jose Saenz Villalobos, Alajuela (CR); Katherin Narvaez Nuñez, Cartago (CR); Cristopher Oviedo Miranda, Alajuela (CR)

(73) Assignee: Boston Scientific Scimed, Inc., Maple Grove, MN (US)

(21) Appl. No.: 19/392,320

(22) Filed: Nov. 18, 2025

Related U.S. Application Data
(60) Provisional application No. 63/722,194, filed on Nov. 19, 2024.

(51) Int. Cl. A61B 18/16; A61B 18/00; A61B 18/12; A61B 18/14

(52) U.S. Cl. CPC A61B 18/16; A61B 18/1206; A61B 18/1492; A61B 2018/00077; A61B 2018/00083; A61B 2018/00184; A61B 2018/00982; A61B 2018/126; A61B 2018/141; A61B 2018/162

(57) ABSTRACT

A medical device may include a first electrically conductive shaft, a second electrically conductive shaft, and a loop. The first electrically conductive shaft may include a first electrically insulating material defining an outer surface of the first electrically conductive shaft. The loop may be in electrical communication with and extend in a distal direction from the first electrically conductive shaft and the second electrically conductive shaft. A portion of the loop may include an electrically conductive circumferential outer surface. The first electrically conductive shaft, the second electrically conductive shaft, and the loop may form a bipolar snare.

### CROSS-REFERENCE TO RELATED APPLICATIONS

This application claims the benefit of U.S. Provisional Patent Application Ser. No. 63/722,194 filed on Nov. 19, 2024, the disclosure of which is incorporated herein by reference.

### TECHNICAL FIELD

The present disclosure pertains to medical devices. More particularly, the present disclosure pertains to medical devices having bipolar electrodes.

### BACKGROUND

Electrosurgical procedures are widely used in modern medicine for cutting, coagulating, and ablating tissue. These procedures utilize electrical current to achieve precise tissue effects. In traditional monopolar electrosurgery, electrical current flows from an active electrode through the patient's body to a dispersive electrode. Bipolar electrosurgery, on the other hand, confines the electrical current to a localized area between two electrodes. Medical devices are used in endoscopic procedures for tissue removal and have been adapted for use with various energy modalities. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including electrosurgical devices, snare devices and/or other medical devices and associated devices.

A first example may be medical device including a first electrically conductive shaft comprising a first electrically insulating material defining an outer surface of the first electrically conductive shaft, a second electrically conductive shaft, and a loop configured to be in in electrical communication with and to extend in a distal direction from the first electrically conductive shaft and the second electrically conductive shaft, a portion of the loop having an electrically conductive circumferential outer surface, and wherein the first electrically conductive shaft, the second electrically conductive shaft, and the loop may form a bipolar snare.

Alternatively or additionally to any of the examples above, the first electrically conductive shaft may include an electrically conductive coil with the first electrically insulating material on an outer surface of the electrically conductive coil.

Alternatively or additionally to any of the examples above, the second electrically conductive shaft may be configured to extend through a lumen defined by the electrically conductive coil and the second electrically conductive shaft may comprise a second electrically insulating material defining an outer surface of a portion of the second electrically conductive shaft extending within the lumen.

Alternatively or additionally to any of the examples above, the medical device may further include an electrically conductive ring coupled with a distal end of the first electrically conductive shaft and the electrically conductive ring may be a portion of a return electrode of the bipolar snare.

Alternatively or additionally to any of the examples above, the electrically conductive ring may define a lumen configured to receive the second electrically conductive shaft and the loop, and the lumen may be defined by an inner surface of a second electrically insulating material.

Alternatively or additionally to any of the examples above, the first electrically conductive shaft may include an electrically conductive coil and the second electrically insulating material may extend into a lumen defined by the electrically conductive coil.

Alternatively or additionally to any of the examples above, the electrically conductive ring may have an electrically conductive outer surface.

Alternatively or additionally to any of the examples above, the loop may be partially insulated to prevent direct electrical contact between the loop and the electrically conductive ring.

Alternatively or additionally to any of the examples above, the first electrically conductive shaft and the second electrically conductive shaft may define the loop.

Alternatively or additionally to any of the examples above, the first electrically insulating material may extend along the loop and define one or openings through which the first electrically conductive shaft is configured to receive current from a portion of the second electrically conductive shaft defining the loop.

Alternatively or additionally to any of the examples above, the loop may include a plurality of wires, one or more of the plurality of wires may extend from the first electrically conductive shaft and has an outer surface defined by a second insulating material defining one or more openings, and the loop may have a distal portion comprising the one or more openings and a proximal portion.

In a further example, a medical device may include a return electrode defining a lumen, an active electrode comprising a control shaft and a loop coupled with the control shaft and configured to translate with the control shaft relative to the lumen, wherein the return electrode may be configured to receive current from the active electrode through tissue of a patient.

Alternatively or additionally to any of the examples above, the return electrode may have a proximal region and a distal region, the proximal region may have an outer surface defined by a first electrically insulating material and the distal region may have an outer surface defined by an electrically conductive material.

Alternatively or additionally to any of the examples above, a second electrically insulating material may define an inner surface of the return electrode extending in a proximal direction from a distal end of the distal region.

Alternatively or additionally to any of the examples above, the medical device may further include an electrically insulating material defining an outer surface of the control shaft and the loop.

Alternatively or additionally to any of the examples above, the control shaft may include one or more wires and the one or more wires may define the loop.

In an further example, a medical device may include an outer shaft defining a lumen, a control shaft configured to extend through the lumen, and a loop coupled with the control shaft and configured to translate with the control shaft relative to the outer shaft, the loop is formed from a plurality of wires, wherein a first wire of the plurality of wires may be configured as an active electrode having an electrically conductive circumferential outer surface defining a portion of the loop and a second wire of the plurality of wires may be configured as a return electrode configured to receive current from the active electrode through tissue of a patient, the second wire having an electrically insulating circumferential outer surface defining a portion of the loop and one or more openings in the electrically insulating circumferential outer surface configured to receive the current from the first wire.

Alternatively or additionally to any of the examples above, the control shaft may include the first wire and the second wire.

Alternatively or additionally to any of the examples above, two or more of the plurality of wires may be configured as the active electrode.

Alternatively or additionally to any of the examples above, the loop may have a proximal portion and a distal portion, the distal portion comprising the one or more openings.

The above summary of some embodiments is not intended to describe each disclosed embodiment or every implementation of the present disclosure. The Figures and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative configuration of a medical system;

FIG. 2 is a schematic side view of a portion of an illustrative configuration of a medical device;

FIG. 3 is a schematic cross-section of the portion of the medical device depicted in FIG. 2;

FIG. 4 is a schematic perspective view of an illustrative configuration a component of a medical device;

FIG. 5 is a schematic perspective cross-section view of the component depicted in FIG. 4;

FIG. 6 is a schematic view of a portion of an illustrative configuration of a medical device with an elongate shaft depicted in cross-section and a control shaft and loop depicted from a side view;

FIG. 7 is a schematic side view of a portion of an illustrative configuration of a medical device;

FIG. 8 is a schematic perspective cross-section view of a portion of an illustrative configuration of a medical device; and

FIG. 9 is a schematic diagram of an illustrative method of using a medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

The present disclosure relates to bipolar snare devices and other medical devices for use in electrosurgical procedures. These devices are designed to provide precise and controlled tissue cutting while confining electrical current to the treatment site. Although the disclosure is discussed primarily with respect to bipolar snare devices, similar concepts may be applied to other suitable medical devices.

Monopolar electrosurgical systems provide electrical current that travels through the patient's body to a dispersive electrode, which can potentially lead to unintended effects on surrounding tissues (e.g., non-target tissue) or implanted medical devices (e.g., pacemakers, etc.). Bipolar systems aim to address these concerns by localizing the current flow between two closely spaced electrodes. For example, in a bipolar system, an active electrode (e.g., a first electrode) may deliver current directly to a target tissue and a return electrode (e.g., a second electrode) may receive the current passing through the target tissue, thus localizing the current applied to a subject to a desired target area.

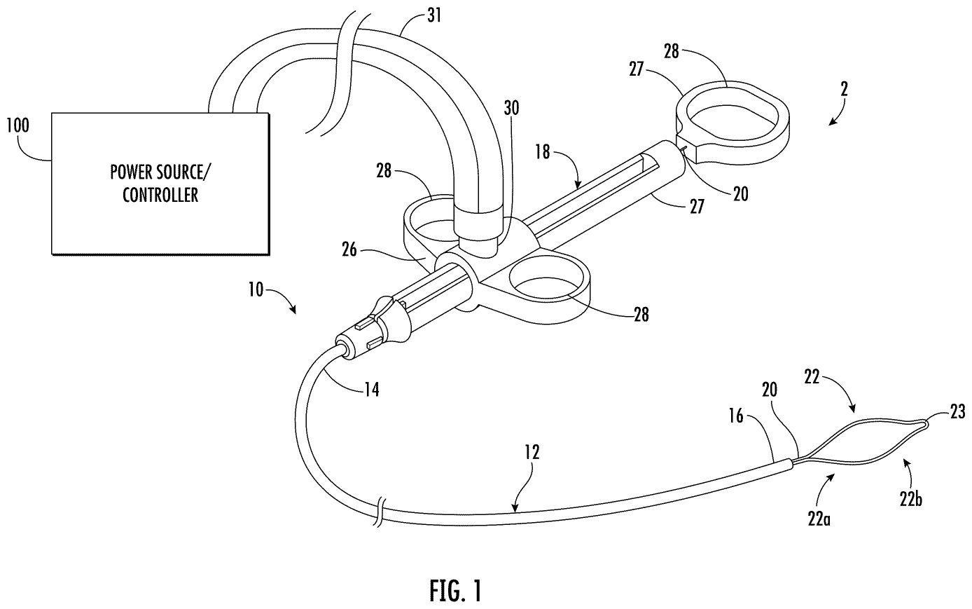

FIG. 1 depicts a schematic configuration of an illustrative medical system 2. The medical system 2 comprises a medical device 10 and a controller 100. In some examples, the medical device 10 may be a snare device and/or other suitable medical device. As depicted in FIG. 1, the medical device 10 may be a bipolar snare device.

The medical device 10 may include a first shaft 12 having a proximal region 14 and a distal region 16 and a second shaft 20. A handle 18 of the medical device 10 may be coupled to the proximal region 14 of the first shaft 12, a proximal region of the second shaft 20, and/or coupled to other suitable components of the medical device 10. Although not depicted in FIG. 1, the medical device 10 and/or the medical system 2 may include an outer shaft or sheath through which the first shaft 12 and/or the second shaft 20 may extend.

The second shaft 20 may extend along the first shaft 12 and/or may be disposed within at least a portion of the first shaft 12. In some examples, the second shaft 20 may be slidable (e.g., rotationally and/or longitudinally translatable) within and/or with respect to the first shaft 12, but other suitable configurations are contemplated. In some examples, the second shaft 20 may be a control shaft.

The medical device 10 may include an end effector configured to be at or proximate a distal end of the first shaft 12 and/or the second shaft 20, such as a loop 22 (e.g., a snare loop and/or other suitable end effector). In some examples, the loop 22 may be coupled with one or both of the first shaft 12 and the second shaft 20. In some examples, the first shaft 12, the second shaft 20, and the loop 22 may form at least part of a bipolar snare.

The loop 22 may be configured to be fixed in one or more dimensions relative to the second shaft 20 and/or adjustable in one or more dimensions relative to the second shaft 20. In one example, the loop 22 may be longitudinally fixed relative to the second shaft 20 (e.g., the loop 22 may be configured to longitudinally translate with the second shaft 20) and rotationally adjust relative to the second shaft 20, such that the loop 22 may be a self-orienting loop. In one example, the loop 22 may be longitudinally and rotationally fixed relative to the second shaft 20 (e.g., the loop may be configured to longitudinally and rotationally translate with the second shaft 20). Other suitable configurations of the loop 22 relative to the second shaft 20 are contemplated.

The handle 18 may have any suitable configuration and may be formed from any suitable material discussed herein or otherwise. In some examples, the handle 18 may be coupled with a proximal end of the second shaft 20 and may operate as a graspable component or portion of medical device 10. For example, the handle 18 may include a first portion 26 coupled with the first shaft 12 and a second portion 27 coupled with the second shaft 20. In some examples, the second portion 27 of the handle 18 may be configured to translate longitudinally and/or rotationally relative to the first portion 26 such that movement of the second portion 27 may result in the second shaft 20 and/or the loop 22 moving in the same longitudinal and/or rotational direction as the second portion 27. For example, the medical device 10 may be configured so that when the second portion 27 and the second shaft 20 are adjusted in a distal direction, the loop 22 may extend distally from the first shaft 12 and/or an outer shaft sheath, where the loop 22 may be in a position for capturing and removing polyps and/or other suitable body tissue. When the second portion 27 and the second shaft 20 are slid or positioned proximally, the loop 22 may be substantially collapsed and at least partially disposed within the first shaft 12 and/or the outer shaft or sheath. Although the handle 18 is discussed with the second portion 27 being adjustable to adjust a positioning of and/or to operate the loop 22, it is contemplated that the first portion 26 and/or other suitable portion of the handle 18 may be adjustable to adjust a position of and/or operate the loop 22.

The handle 18 may include a number of structural features. For example, the first portion 26 and/or the second portion 27 of the handle 18 may include one or more finger holes 28 to help assist in grasping and/or positioning the handle 18. In one example, the first portion 26 of the handle 18 may include two finger holes 28 each for receiving a finger of a user and the second portion 27 of the handle 18 may include a single finger hole 28 for receiving a finger (e.g., a thumb or other suitable finger) of a user to facilitate adjusting or otherwise moving the second portion 27 with and/or relative to the first portion 26 of the handle 18. Alternatively, the second portion 27 of the handle 18 may include two finger holes 28 and the first portion 26 of the handle may include a single finger hole 28. Other suitable features and/or configurations of the handle 18 are contemplated.

The first portion 26 of the handle 18 may include a port 30. The port 30 may be used, for example, as an access point to apply and/or deliver electrosurgical current to the second shaft 20 and/or snare loop 22 and receive a return current. Other suitable configurations of the port 30 are contemplated.

Although not depicted in FIG. 1, one or more outer shafts or sheaths or other suitable elongate tubes may extend over the first shaft 12 and/or the second shaft 20. In some examples, the one or more outer shafts or sheaths or other suitable elongate tubes may be guide sheaths, insulating sheaths, and/or other suitable types of shafts or sheaths. In one example, when the first shaft 12 is configured as one or more wires extending along the second shaft 20 (e.g., as discussed in greater detail herein), the outer shaft or sheath may facilitate closing the loop 22 around tissue when the first shaft 12 and/or the second shaft 20 are retracted in a proximal direction relative to the outer shaft or sheath. Other suitable configurations of the sheath are contemplated.

The first shaft 12 may have any suitable configuration. In some examples, the first shaft 12 may be an electrically conductive shaft and/or an electrically insulative shaft, but other suitable configurations are contemplated. For example, the first shaft 12 may have one or more layers of electrically conductive material, one layers of electrically insulative materials, or one or more layers of electrically conductive materials and one or more layers of electrically insulative materials. In one example, an outer layer defining an outer surface of the first shaft 12 may a layer of insulative material and an inner layer of the first shaft 12 may be an electrically conductive material. The layer of conductive material of the first shaft 12 may define an inner surface of the first shaft 12 or a core material of the first shaft 12. In some examples, an electrically conductive first shaft 12 may be configured as a return electrode for the medical device 10, but other suitable configurations are contemplated.

In some example configurations, the first shaft 12 may be tubular and configured to bend along the length of the first shaft 12 as the first shaft 12 passes through one or more body cavities or lumens. In one example, when the first shaft 12 is formed from or as a tube, the first shaft 12 may define a lumen configured to receive the second shaft 20 and/or the loop 22. The first shaft 12 configured as a tube may include an inner layer that is electrically conductive (e.g., a first conductive material) and an outer layer that electrically insulative (e.g., a first insulating material). Alternatively or additionally, the first shaft 12 may include an inner layer of material defining the lumen that is electrically insulative, such that the layer of electrically conductive material may be between two layers of insulative material for at least a portion of an entire length of the first shaft 12.

The layer of electrically conductive material of a tubular configuration of the first shaft 12 may have any suitable configuration. For example, the layer of electrically conductive material of a tubular configuration of the first shaft 12 may be formed as an electrically conductive tube, cut tube, coil or coiled tube, spring tube, length of wire, length of ribbon of wire, braided wire, helical wire, and/or one or more other suitable configurations. Although other suitable materials may be utilized for the layer of electrically conductive material of the first shaft 12, the electrically conductive material may be a metal, a stainless steel, and/or other suitable material. Other suitable configurations of the first shaft 12 configured as a tube are contemplated.

In addition to or as an alternative to the electrically conductive first shaft 12 being formed from or as a tubular structure, the electrically conductive first shaft 12 may be formed from one or more wires extending along the second shaft 20 and configured to bend along the length of the first shaft 12 as the first shaft 12 passes through one or more body lumens. In one example, when the first shaft 12 is formed from or as one or more wires or otherwise a solid structure, the first shaft 12 may be braided with, twisted with, and/or entwined with one or more wires of the second shaft 20. In some examples, the braiding, twisting, or entwining of the first shaft 12 and the second shaft 20 may form a helical configuration. When the electrically conductive first shaft 12 is configured as a wire, the first shaft 12 may include an inner layer or core that is electrically conductive and an outer layer that electrically insulative. Other suitable configurations of the first shaft 12 configured as a wire are contemplated.

The second shaft 20 may have any suitable configuration. In some examples, the second shaft 20 may be an electrically conductive shaft and/or an electrically insulative shaft. For example, the second shaft 20 may have one or more layers of electrically conductive materials, one or more layers of electrically insulative materials, or one or more layers of electrically conductive materials and one or more layers of electrically insulative materials. In one example, an outer layer defining an outer surface of the second shaft 20 may be an electrically insulative material and an inner layer of the second shaft 20 may be an electrically conductive material. The outer layer of electrically insulative material may be provided to prevent or mitigate the likelihood of creating a short with the first shaft 12. In some example configurations of the second shaft 20, the layer of electrically conductive material of the second shaft 20 may be an inner core layer of material and the layer of electrically insulative material may be an outer layer configured to extend along and/or face a layer or surface of electrically conductive material of the first shaft 12. In one example configuration, when the medical device 10 is a bipolar medical device, an electrically conductive second shaft 20 may be configured as or part of an active electrode for the medical device 10, but other suitable configurations are contemplated. In some examples, the second shaft 20 may be or may act as a control shaft or wire (e.g., formed from one or more wires) such that manipulation of the second shaft 20 may manipulate the loop 22 in one or more dimensions.

The second shaft 20 may be formed from one or more wires extending along the first shaft 12, but other configurations of the second shaft 20 relative to the first shaft 12 are contemplated. In one example, the second shaft 20 may be formed from a plurality of wires. In one example, when the second shaft 20 is formed from or as one or more wires or otherwise a solid structure, the second shaft 20 may be braided with, twisted with, or entwined one or more wires of the first shaft 12. In some examples, the braiding, twisting, or entwining of the first shaft 12 and the second shaft 20 may form a helical configuration, but other suitable configurations are contemplated. When the second shaft 20 is configured as one or more wires and extends along the first shaft 12 having a layer of electrically insulating material facing an outer surface of the second shaft 20, the layer of electrically insulating material of the second shaft 20 may be omitted. Other suitable configurations of the second shaft 20 configured as a wire are contemplated.

The loop 22 may have any suitable configuration configured to be in electrical communication with and extend in a distal direction from or with respect to the first shaft 12 and/or the second shaft 20. The loop 22 may have any suitable configuration configured to be part of the second shaft 20 or to be in electrical communication with the second shaft 20 to act as an active electrode or to be part of an active electrode that includes the second shaft 20. Additionally, the loop 22 may include or may be configured to be in electrical communication with the first shaft 12 acting as a return electrode.

The loop 22 may have any suitable shape and/or size configured to extend around body tissue when extended distally from the first shaft 12 and/or an outer shaft or sheath and grasp body tissue as the loop is adjusted proximally into the first shaft 12 and/or the outer shaft or sheath. In some examples, the loop 22 may have a proximal portion 22a and a distal portion 22b. In one example, the proximal portion 22a may include a section that radially widens in a distal direction and the distal portion 22b may include a section that radially narrows in a distal direction to a distal-most location or tip 23. Other suitable configurations of the shape and/or size of the loop 22 are contemplated.

The loop 22 may be formed from one or more wires. In some examples, the loop may be formed from one or more wires of the first shaft 12, one or more wires in communication with the first shaft 12, one or more wires of the second shaft 20, one or more wires in communication with the second shaft 20, one or more other wires, and/or a combination of wires in communication with first shaft 12 and/or the second shaft 20. The one or more wires of the loop 22, when included, may be selectively insulated from one another at one or more locations, as desired.

The loop 22 may have one or more portions having an electrically conductive outer surface that forms at least part of the active electrode and are configured to engage body tissue of a subject. In some examples, the loop 22 may have one or more portions with an electrically conductive outer surface that continuously extends around a circumference of the wire. In some examples, the loop 22 may have one or more wires that are bare of any electrically insulating material with an electrically conductive outer surface around an entirety of the loop 22. In some examples, the loop 22 may have one or more wires that have an electrically insulating outer surface over at least a portion of loop 22.

The first shaft 12, the handle 18, the second shaft 20, the loop 22, and/or other components of the medical device 10 may be formed from any suitable materials. Example suitable materials include, but are not limited to, polymers, metals, metal alloys, metal-polymer composites, and/or other suitable materials. Some example suitable polymers include, but are not limited to, polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, a polyether-ester elastomer such as ARNITEL® available from DSM Engineering Plastics), polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example, available under the trade name PEBAX®), silicones, polyethylene (PE), Marlex high-density polyethylene, Marlex low-density polyethylene, linear low density polyethylene (for example, REXELL®), polyethylene terephthalate (PET), polyetheretherketone (PEEK), polyimide (PI), polyetherimide (PEI), polyphenylene sulfide (PPS), polyphenylene oxide (PPO), polysulfone, nylon, perfluoro(propyl vinyl ether) (PFA), other suitable materials, or mixtures, combinations, copolymers thereof, polymer/metal composites, and the like. Some example suitable metals and/or metal alloys include, but are not limited to, stainless steel, such as 304V, 304L, and 316L stainless steel; nickel-titanium alloy such as linear-elastic or super-elastic nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like.

The port 30 may be electrically coupled with the first shaft 12, the second shaft 20, and an electrical connector 31, where the first shaft 12 may be or may include the return electrode or other suitable component to collect return current from body tissue of a subject and the second shaft 20 may be or may include the active electrode or other suitable component configured to provide electrosurgical current to the loop 22 and/or the body tissue of the subject. The electrical connector 31 may be coupled with one or more power sources or controllers 100 configured to supply electrosurgical current to the medical device 10 and/or receive return current from the medical device 10 to create a bipolar circuit. The power source or controller 100 may be configured to facilitate operation of the medical device 10.

The power source or controller 100 may be and/or may include any suitable computing device configured to process data of or for the medical device 10. In some cases, the power source or controller 100 may be configured to apply power (e.g., current) to the medical device 10 in response to one or more user inputs, control signals, conditions of the medical device, conditions of the subject, a schedule, and/or one or more other prompts. Further, one or more components of the medical device 10 may incorporate one or more computing devices similar to or having components similar to the power source or controller 100.

The illustrative power source or controller 100 may include, among other suitable components, one or more processors, memory, and/or one or more I/O units. Example other suitable components of the power source or controller may include, but are not limited to, communication components, a touch screen, selectable buttons, a housing, an electrical connection to line power, and/or other suitable components of a power source or controller. As discussed above, one or more components of the power source or controller 100 may be separate from the components of the medical device 10 and/or incorporated into the components of the medical device 10.

The processor of the power source or controller 100 may include a single processor or more than one processor working individually or with one another. The processor may be configured to receive and execute instructions, including instructions that may be loaded into memory. Example components of the processor may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory of the power source or controller 100 may include a single memory component or more than one memory component each working individually or with one another. Example types of memory may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory may be or may include a non-transitory computer readable medium. The memory may include instructions stored in transitory and/or non-transitory state on a computer readable medium that may be executable by the processor to cause the processor to perform one or more of the methods and/or techniques, or a portion thereof, described herein.

The I/O units of the power source or controller 100 may include a single I/O component or more than one I/O component each working individually or with one another. Example I/O units may be or may include any suitable types of communication hardware and/or software including, but not limited to, communication ports configured to communicate with electronic or electrical components of the medical device 10 and/or with other suitable devices or systems. Example types of I/O units may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units.

The medical system 2 may include a user interface configured to communicate with the power source or controller 100 via one or more wired or wireless connections. The user interface may include one or more display devices, one or more input devices, one or more output devices, and/or one or more other suitable features.

The display device, when included with the user interface, may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s), when included with the user interface, may be and/or may include any suitable components and/or features for receiving user input via the user interface. Example input device(s) include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components), and/or other suitable input devices.

The output device(s), when included with the user interface may be and/or may include any suitable components and/or features for providing information and/or data to users and/or other computing components. Example output device(s) include, but are not limited to, displays, speakers, vibration systems, tactile feedback systems, optical outputs, cables, lights, and/or other suitable output devices.

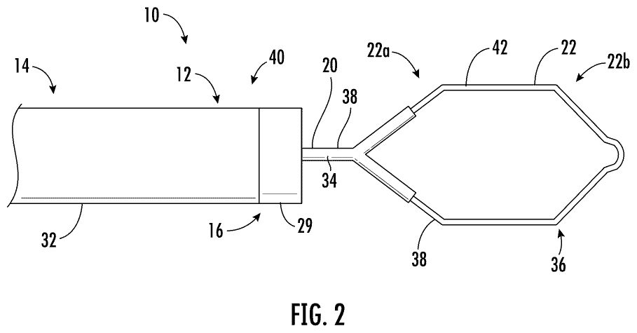

FIG. 2 schematically depicts a portion of an illustrative bipolar configuration of the medical device 10. For example, the second shaft 20 and/or the loop 22 of the medical device 10 may be configured as an active electrode 36 (e.g., a first electrode) that provides an electrosurgical current from the power source or controller 100 (not shown in FIG. 2) to body tissue of a subject and the first shaft 12 and/or a distal component 29 may be configured as a return electrode 40 (e.g., a second electrode) between the active electrode 36 at the loop 22 and the power source or controller 100. In some examples, the return electrode 40 may be configured to receive electrical current from the body tissue of the subject that was applied to the body tissue by the active electrode 36. The first shaft 12 and/or the distal component 29 may be configured to adjustably receive the second shaft 20 and/or at least a portion of the loop 22.

As depicted in FIG. 2, the first shaft 12 acting as the return electrode 40 may have any suitable configuration defining a lumen configured to receive the second shaft 20. In some examples, the first shaft 12 may be defined by an electrically conductive tubular structure with a first shaft electrically insulating material 32 on at least a portion of an outer surface of the electrically conductive tubular structure. Alternatively or additionally, the first shaft 12 may be defined by an electrically insulating tubular structure formed of the first shaft electrically insulating material 32 with one or more electrically conductive members (e.g., wires, ribbons, shafts, etc.) embedded in or otherwise secured to the electrically insulating tubular structure and extending from a distal end of the electrically insulating tubular structure to a proximal end of the electrically insulating tubular structure so as to be configured to receive current from body tissue and to be in communication with the power source or controller 100.

The first shaft electrically insulating material 32 may be on any portion of the electrically conductive tube when the electrically conductive tube is utilized, as in the configuration of the medical device 10 depicted in FIG. 2. For example, the first shaft electrically insulating material 32 may only be on the outer surface of the electrically conductive tubular structure, may only be on an inner surface of the electrically conductive tubular structure, or may be on an outer surface and an inner surface of the electrically conductive tube. In one example configuration, the first shaft electrically insulating material 32 may extend over the outer surface of the electrically conductive tubular structure (e.g., as an insulating jacket, etc.) and define an outer surface of the first shaft 12. The first shaft electrically insulating material 32 may be a tube of the first shaft electrically insulating material 32 that is reflowed over the electrically conductive tubular structure, but other suitable techniques for applying the first shaft electrically insulating material 32 onto the electrically conductive tubular structure may be utilized.

Although other configurations are contemplated, the embodiment of the medical device 10 depicted in FIG. 2 may include an electrically conductive tubular structure, as discussed, that is configured to adjustably receive the second shaft 20 and/or the loop 22. Any suitable electrically conductive tubular structure defining a lumen configured to adjustably receive the second shaft 20 and/or the loop 22 and that is sufficiently flexible to traverse body lumens may be utilized. In one example, an electrically conductive tubular coil may be utilized. When an electrically conductive tubular coil is utilized, the electrically conductive tubular coil may have a bare inner surface or an electrically insulated inner surface and an electrically insulated outer surface.

When the first shaft 12 has a bare inner surface defining a lumen (e.g., an electrically conductive inner surface), at least a portion of the second shaft 20 traversing the lumen of the first shaft 12 may have an outer layer of a second shaft electrically insulating material 34, as depicted for example in FIG. 2, to prevent or mitigate electricity leakage and/or unintentional electrical communication (e.g., direct electrical contact or communication that may cause a short circuit) between the second shaft 20 and the first shaft 12 within the lumen. In some examples, at least a portion of the loop 22 configured to be received at or within the lumen of the first shaft 12 may include the second shaft electrically insulating material 34 and/or other suitable electrically insulating material to prevent or mitigate unintentional electrical communication (e.g., direct electrically communication) between the loop 22 and the first shaft 12 when the loop 22 is withdrawn into the lumen of the first shaft 12. When the first shaft 12 has an electrically insulated inner surface, the second shaft electrically insulating material 34 may be omitted.

The active electrode 36 of the medical device 10 may be exposed to body tissue at the loop 22. In some examples, the exposed active electrode 36 may be a portion of one or more wires 38 defining the loop 22 and/or the second shaft 20 that has an exposed electrically conductive outer surface of a layer 42 of an electrically conductive material (e.g., an electrically conductive core and/or other suitable layer) electrically coupled with the power source/controller 100. For example, as depicted in FIG. 2, the exposed active electrode 36 may be a portion of the wire 38 extending in a distal direction from the second shaft electrically insulating material 34 at the loop 22. Although the exposed portion of the active electrode 36 is depicted in FIG. 2 as extending from the proximal portion 22a to the distal portion 22b of the loop 22, it is contemplated that the active electrode 36 is exposed only at the proximal portion 22a of the loop 22 or only at the distal portion 22b of the loop 22. In one example, the active electrode 36 may only be exposed at the distal portion 22b of the loop such that the exposed active electrode 36 focuses electrosurgical current passing therethrough at or proximate to a location at which body tissue is to be secured within the loop 22, but other suitable configurations are contemplated.

The distal component 29 may be located at a distal end of the first shaft 12 and may be or may form part of the return electrode 40. The distal component 29 may be part of the first shaft 12 or may be separate from first shaft 12 and electrically coupled with the first shaft 12. In some examples, the distal component 29 may form or define an electrically conductive outer surface at the distal region 16 of the first shaft 12 and the first shaft insulating material 32 may form or define an electrically insulating outer surface at the proximal region 14 of the first shaft 12. In some examples, the distal component 29 may form a distal-most portion of the return electrode 40 configured to receive electrical current from body tissue of the subject that is engaged by the loop 22 and the active electrode 36 applying electrosurgical current to the engaged body tissue.

The distal component 29 may have any suitable configuration configured to receive electrical current from body tissue of the subject applied thereto by the active electrode 36. For example, the distal component may be or may include one or more electrical contacts, a ring, a cap, a bushing, and/or one or more other suitable electrically conductive components positioned at or proximate the distal end of the first shaft 12. In one example and as depicted in FIG. 2., the distal component 29 may be an electrically conductive ring or bushing of or coupled with the first shaft 12, but other suitable configurations are contemplated. When the distal component 29 is configured as an electrically conductive ring or bushing, the distal component 29 may include an electrically conductive outer surface configured to receive current from the body tissue and act as or be part of the return electrode 40.

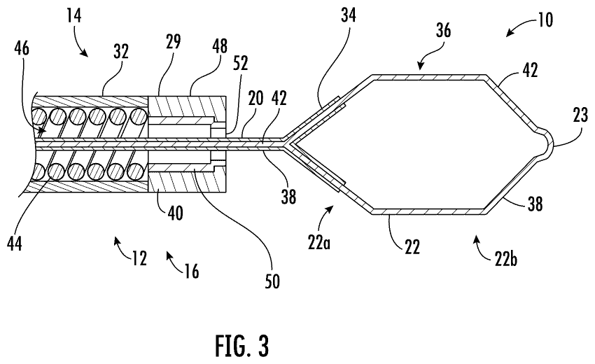

FIG. 3 depicts a schematic cross-section view of the distal portion of the illustrative bipolar configuration of the medical device 10 depicted in FIG. 2. As depicted in FIG. 3, the first shaft 12 may define a lumen 46 and the second shaft 20 may extend through the lumen 46. In some examples, the lumen 46 may be configured to adjustably receive the second shaft 20 and/or the loop 22 such that the second shaft 20 and/or the loop 22 may be rotationally and/or longitudinally translatable relative to the first shaft 12.

The first shaft 12 may include an elongate electrically conductive tubular structure 44 defining the lumen 46 and the first shaft insulating material 32 forming an insulating outer surface or layer of the first shaft 12 on an outer surface of the electrically conductive tubular structure 44. Although other suitable configurations are contemplated, the electrically conductive tubular structure 44 may be an electrically conductive coil tube with an electrically conductive inner surface (e.g., an inner surface that is not electrically insulated). In some examples, the electrically conductive tubular structure 44 may form at least part of the return electrode 40. The electrically conductive tubular structure 44 may be formed form a metal material, such as stainless steel for example, and/or one or more other suitable electrically conductive materials.

The distal component 29 of or coupled with the first shaft 12 may further define the lumen 46 configured to receive the second shaft 20 and/or the loop 22. As depicted in FIG. 3, the distal component 29 may have one or more layers 48 of electrically conductive material and one or more layers 50 of electrically insulating material. In some examples, the layer 48 of electrically conductive material may act as and/or form an entirety of or at least part of the return electrode 40. In some examples, the layer 50 electrically insulating material of the distal component 29 may entirely or at least partially define the lumen 46 and a distal opening 52 of the first shaft 12. The layer 50 of electrically insulating material may be configured to prevent an exposed electrically conductive layer 42 of the one or more wires 38 at the loop 22 that forms at least a portion of the active electrode 36 from directly electrically contacting an electrically conductive portion of the return electrode 40. Although not depicted in FIG. 3, the electrically insulating layer 50 of the distal component 29 may extend into the electrically conductive tubular structure 44 (e.g., extend from the distal region 16, such as from the distal end of the distal region 16, in a proximal direction to the proximal region 14) to further prevent the active electrode 36 at the loop 22 from directly electrically contacting an electrically conductive surface of the return electrode 40 when the loop 22 is at least partially positioned within the lumen 46.

In some examples, the distal component 29 may be mechanically and/or electrically coupled with the electrically conductive tubular structure 44 to form at least a portion of the return electrode 40. For example, the layer 48 of electrically conductive material of the distal component 29 may be mechanically and/or electrically coupled with the electrically conductive tubular structure 44 using any suitable technique including, but not limited to, welding, soldering, crimping, and/or other suitable mechanical and electrical coupling techniques.

As depicted in FIG. 3, the second shaft 20 and the loop 22 may be formed from the same wire 38, but other suitable configurations in which the second shaft 20 and/or the loop 22 are formed from different wires 38 are contemplated. The wire 38 may include the layer 42 of electrically conductive material extending along the second shaft 20 and the loop 22, with the second shaft electrically insulating material 34 extending along the second shaft 20 and the proximal portion 22a of the loop 22. The second shaft electrically insulating material 34 may prevent the portions of the layer 42 of electrically conductive material from directly electrically contacting the inner surface of the electrically conductive tubular structure 44 (e.g., may prevent direct electrical contact between the active electrode 36 and the return electrode 40) when the second shaft 20 and/or the loop 22 are positioned within the first shaft 12.

The layer 42 of electrically conductive material of the wire 38 may be exposed at the loop to form a portion of the active electrode 36 configured to apply the electrosurgical current to body tissue of the subject. As depicted in FIG. 3, the electrically conductive layer 42 may be exposed at the distal portion 22b and the proximal portion 22a of the loop 22, but other suitable configurations are contemplated.

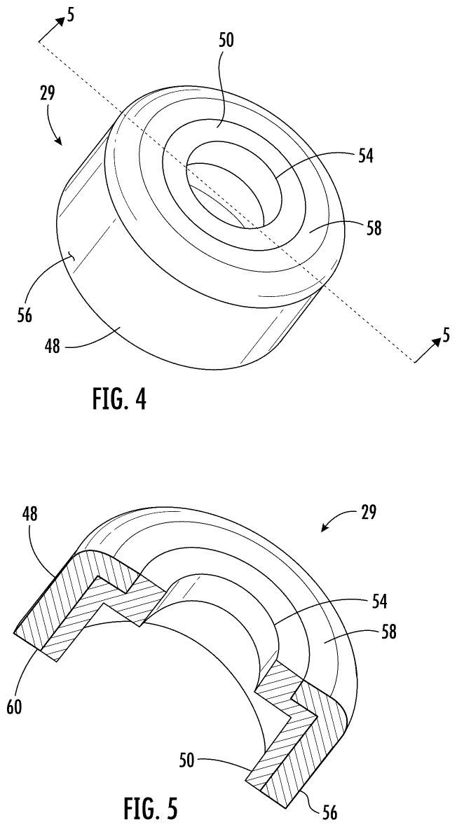

FIG. 4 depicts a schematic perspective view of an illustrative configuration of the distal component 29. As depicted in FIG. 4, the distal component 29 may be ring-shaped with a central opening 54 configured to define the distal opening 52 (not shown in FIG. 4) of the first shaft 12. In one example, the configuration of the distal component 29 may include a radial outer circumference 56, a distal facing end 58, and a transition between the radial outer circumference and the distal facing end 58, where the transition may be a taper, a curve, and/or other suitable configuration configured to facilitate advancing the first shaft 12 within a lumen. In one example, the configuration of the distal component 29 depicted in FIG. 2 may be a bushing. Other suitable configurations of the distal component 29 are contemplated.

A discussed, the distal component 29 may include the layer 48 of electrically conductive material and the layer 50 of electrically insulating material. The layer 48 of electrically conductive material may form an outer surface of the distal component 29 along at least part of a radial outer circumference 56 of the distal component 29 and/or at least part of a distal facing end 58 of the distal component 29. In one example, the layer 48 of electrically conductive material may form an outer surface of the distal component 29 along an entirety of the radially outer circumference 56 of the distal component 29 and a portion of the distal facing end 58 of the distal component, as depicted in FIG. 4. The layer 50 of electrically insulating material may form an inner surface of the distal component 29 and/or an outer surface of the distal facing end 58 of the distal component 29. In some examples, the layer 50 of electrically insulating material may form or define an entirety of an inner surface of the distal component 29 and a portion of the distal facing end 58, as depicted in FIG. 4. In one example, the layer 50 of electrically insulating material may be configured to define the central opening 54 of the distal component 29 and a portion of the lumen 46 (not shown in FIG. 4) of the first shaft 12, but other suitable configurations are contemplated. The layer 50 of electrically insulating material of the distal component 29 may be configured to define a physical and electrical barrier between conductive surfaces of the active electrode 36 and the return electrode 40 to prevent those conductive surfaces from physically contacting one another as the second shaft 20 and/or the loop 22 are adjusted relative to the first shaft 12 and within the lumen 46 and distal opening 52.

FIG. 5 depicts a schematic cross-section view of the distal component 29, taken along line 5-5 of FIG. 4. As depicted in FIG. 5, the layer 48 of electrically conductive material may form an outer surface of the distal component 29 along the radial outer circumference 56 and the distal facing end 58, while the layer 50 of electrically insulating material may form an inner surface of the distal component 29 and an outer surface of at the distal facing end 58 of the distal component 29.

Although the layer 50 of electrically insulating material is depicted in FIG. 5 as defining an entirety of the inner surface of the distal component 29 (e.g., where the inner surface of the distal component is symmetric about line 5-5 in FIG. 4), it is contemplated that the layer 50 electrically insulating material may not extend to a proximal end 60 of the distal component 29. When the layer 50 of electrically insulating material does not extend to the proximal end 60 of the distal component 29, the layer 50 of electrically insulating material may taper radially inward from a proximal-most end of the layer 50, but other suitable configurations are contemplated. Additionally or alternatively, the layer 50 electrically insulating material may extend beyond the proximal end 60 of the distal component to form the layer 50 of electrically insulative material on an inner surface of the electrically conductive tubular structure 44.

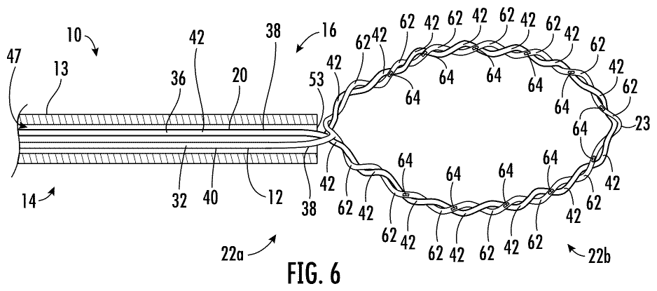

FIG. 6 depicts a schematic view of an illustrative configuration of the medical device 10. As depicted in FIG. 6, an outer shaft or sheath 13 of the medical device 10 is depicted in cross-section, while first shaft 12, the second shaft 20, and the loop 22 are depicted from a side view. In some examples, the first shaft 12 and the second shaft 20 may form a control wire or control shaft from which the loop 22 may extend.

The outer shaft or sheath 13 of the medical device 10 depicted in FIG. 6 may have any suitable configuration that defines a lumen 47 and a distal opening 53, such that the outer shaft or sheath 13 may receive the first shaft 12, the second shaft 20, and/or the loop 22 within the lumen 47 and the distal opening 53 and/or such that the first shaft 12, the second shaft 20, and/or the loop 22 may rotationally and/or longitudinally translate relative to the lumen 47 and the distal opening 53. In some examples, the sheath 13 may include and/or be defined by one or more electrically insulating materials, but other suitable configurations of the sheath 13 are contemplated.

The second shaft 20 may include or define the active electrode 36 and first shaft 12 may include and/or define the return electrode 40. In some examples, the active electrode 36 and the return electrode 40 may be configured to extend along one another from a proximal region to a distal region of the second shaft 20 and/or the first shaft 12 and/or through the loop 22. For example, the active electrode 36 and the return electrode 40 may be configured to extend from the proximal region to the distal region of the second shaft 20 and/or the first shaft 12 parallel to one another, braided, twisted, or entwined with one another, and/or may be in one or more other suitable longitudinally extending relationships with one another. In one example and as depicted for example in FIG. 6, the second shaft 20 may include the active electrode 36 extending parallel with the first shaft 12 including the return electrode 40 between the proximal and distal regions of the second shaft 20 and/or the first shaft 12. The second shaft 20 and the active electrode 36 may be coupled with the first shaft 12 and the return electrode 40 in any suitable manner, but this is not required and the second shaft 20 may be adjustable relative to first shaft 12 within the sheath 13.

The active electrode 36 at the second shaft 20 may be formed from one or more of the wires 38. In some examples, the active electrode 36 at the second shaft 20 may be entirely or at least partially uninsulated such that the layer 42 electrically conductive material of the one or more wires 38 is exposed along the second shaft 20 and/or the second shaft insulating material 34 may be applied to the layer 42 of the one or more wires 38 of the active electrode 36. In one example configuration, the active electrode 36 at the second shaft 20 may include a single wire 38 with the layer 42 of electrically conductive material of the single wire 38 exposed or otherwise uninsulated, as depicted for example in FIG. 6.

The return electrode 40 at the first shaft 12 may be formed from one or more of the wires 38. In some examples, the return electrode 40 at the first shaft 12 may be entirely or at least partially uninsulated such that the layer 42 of electrically conductive material of the one or more wires 38 is exposed along the first shaft 12 and/or the first shaft insulating material 32 may be applied to the layer 42 of one or more wires 38 of the return electrode 40. In one example configuration, the return electrode 40 at the first shaft 12 may include a single wire 38 with the single wire 38 having the first shaft insulating material 32 defining an outer surface along a length thereof, as depicted for example in FIG. 6. In addition to and/or as an alternative to the first shaft insulating material 32 being part of the one or more wires 38 of the first shaft 12, the first shaft insulating material 32 may be configured to longitudinally extend between the wire 38 of the active electrode 36 and the wire 38 of the return electrode 40 to electrically isolate the active electrode 36 and return electrode 40 from one another.

The loop 22 may be formed in any suitable manner. In some examples, the loop 22 may be configured to grasp body tissue and coupled with the second shaft 20 and/or the first shaft 12. In one example, the loop 22 may be formed from a plurality of wires 38 that are braided together, twisted together, and/or otherwise entwined with one another. In some examples, the combined wires 38 of the loop 22 may form a single wire loop.

One or more wires 38 of the plurality of wires 38 defining and/or forming the loop 22 may have an electrically insulating outer surface defined by a loop insulating material 62 and may be in communication with the one or more wires 38 forming the return electrode 40 of or along the first shaft 12. The wire 38 including the loop insulating material 62 may be the same (e.g., the first shaft 12 may at least partially define the loop 22) or different wire 38 forming the return electrode 40 of or along the first shaft 12. When the wire 38 including the loop insulating material 62 is different than the wire 38 forming the return electrode 40 of or along the first shaft 12, the wire 38 forming the return electrode 40 of the first shaft 12 may be coupled with the wire 38 with the loop insulating material 62 using suitable mechanical and electrically conductive techniques (e.g., welding, soldering, crimping, etc.) The loop insulating material 62 may be the same insulating material forming the first shaft insulating material 32 and/or different insulating material. The wire 38 having the outer surface defined by the loop insulating material 62 may form the return electrode 40 at the loop 22.

One or more of the wires 38 defining and/or forming the loop 22 may be uninsulated such that the outer surface of the wires 38 may be defined by the layer 42 of electrically conductive material. The uninsulated wires 38 of the loop 22 may be in electrical communication with the active electrode 36 along the second shaft 20 and may form the active electrode 36 at the loop 22. The one or more wires 38 forming the active electrode 36 at the loop 22 may be the same wire 38 (e.g., the second shaft 20 may at least partially define the loop 22) or different wires 38 than the wire(s) 38 forming the active electrode 36 at the second shaft 20. When the one or more wires 38 forming the active electrode 36 at the loop 22 are different than the one or more wires 38 forming the active electrode 36 of or along the second shaft 20, the one or more wires 38 with forming the active electrode 36 of the second shaft 20 may be coupled with the wire 38 forming the active electrode 36 at the loop 22 using suitable mechanical and electrically conductive techniques (e.g., welding, soldering, crimping, etc.)

The wire(s) 38 of the loop 22 having an electrically insulating outer surface defined by the loop insulating material 62 may include one or more windows or openings 64 and/or other portions that electrically expose a conductive surface of the wires 38 to electrical current from the active electrode 36 of the loop 22. In some examples, openings 64 in the loop insulating material 62 may be configured to receive electricity from the active electrode 36 via body tissue received within the loop 22. As such, the openings 64 may be located on the return electrode 40 at locations of the loop 22 intended to contact and/or be proximate the body tissue received within the loop 22.

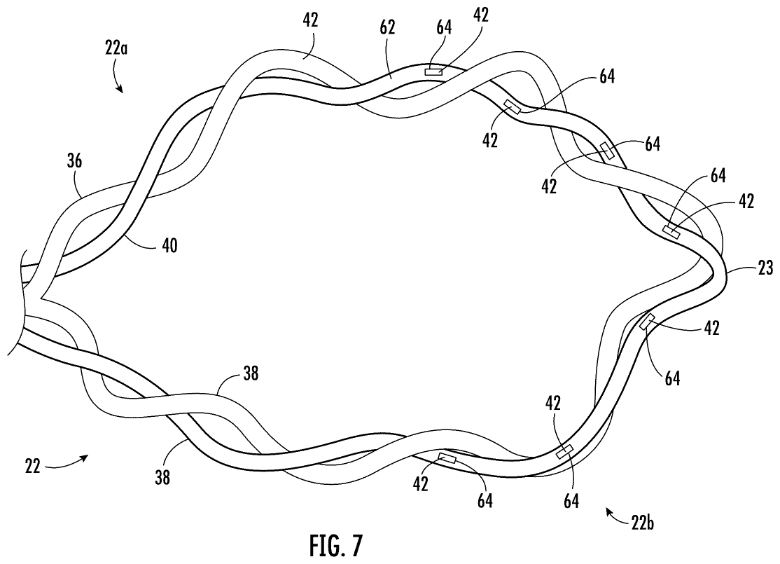

FIG. 7 is a schematic side view of an illustrative configuration of the loop 22 of the medical device 10. As depicted in FIG. 7, the loop 22 may include a plurality of wires 38 braided, twisted, or intertwined with one another. Although only two wires are depicted in FIG. 7, other suitable numbers of wires 38 may be utilized to form the loop 22 such that the loop 22 may be adjustable to engage, receive, cut, and/or release body tissue.

The plurality of wires 38 of the loop 22 may include one or more wires 38 forming the active electrode 36 with the electrically conductive layer 42 forming an outer surface of all or at least a portion of the wire(s) 38 forming the active electrode 36. The plurality of wires 38 include one or more wires 38 forming the return electrode 40 with the loop insulating material 62 forming an outer surface of all or substantially all of the outer surface of the one or more wires 38 forming the return electrode 40. The one or more wires 38 forming the return electrode 40 may include one or more of the openings 64 configured to expose a surface of the layer 42 of electrically conductive material of the wire 38 that is configured to receive electrical current from the active electrode 36 via body tissue within the loop 22.

As discussed, the one or more openings 64 in the loop insulating material 62 may be configured to be proximate to and/or to contact body tissue received within the loop 22. In some examples and as depicted in FIG. 7, the openings 64 may be located at the distal portion 22b of the loop 22. Other suitable positioning of the openings 64 along the return electrode 40 at the loop 22 are contemplated.

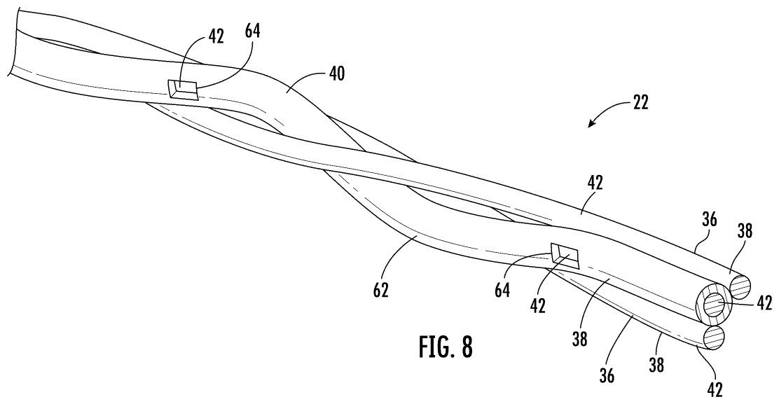

FIG. 8 depicts a schematic perspective cross-section view of a section of the loop 22 including a plurality of wires 38. As discussed, the plurality of wires 38 forming the loop 22 may be braided, twisted, and/or otherwise entwined with one another. In some examples, the wires 38 may form the active electrode 36 and the return electrode 40 at the loop 22.

As depicted in FIG. 8, the active electrode 36 may be formed from a plurality of uninsulated wires 38 having the layer 42 of electrically conductive material defining an outer surface of the wire 38 (e.g., the uninsulated wires may have an electrically conductive circumferential outer surface). The return electrode 40 may be formed form one or more wires 38 (e.g., a single wire 38 in the configuration of the loop 22 depicted in FIG. 8) having the loop insulating material 62 defining an outer surface of the wire(s) 38 (e.g., defining an electrically insulating circumferential outer surface) with the layer 42 of electrically conductive material as a core or within the loop insulating material 62. The openings 64 in the loop insulating material 62 may form electrically conductive surfaces of the layer 42 of electrically conductive material within the loop insulating material 62 that are configured to receive current from the active electrode 36 via body tissue captured within the loop 22 and/or other body tissue proximate the loop to form a bipolar configuration of the loop 22.

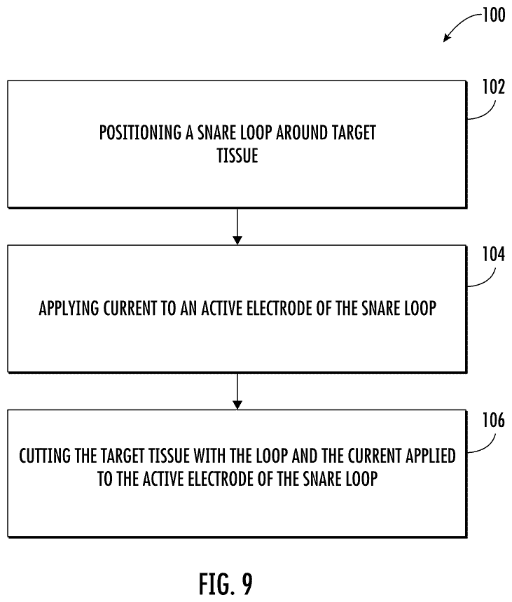

FIG. 9 depicts a schematic diagram of an illustrative method 200 of using a medical device configured as a bipolar snare, as discussed herein and/or otherwise. In operation, the bipolar snare may be inserted into a body cavity of a subject so that the snare loop may be proximate a target tissue (e.g., a polyp, etc.)

Once in the body cavity, the method 200 may include positioning 202 the snare loop around the target tissue within the body cavity or a body lumen of the subject. In some examples, retracting the snare loop into shaft or sheath may constrict the snare loop around the target tissue. A current may be applied 204 to an active electrode of the snare loop in which the target tissue is located. In some examples, the target tissue within the snare loop may contact the snare loop and complete a circuit between the active electrode and a return electrode at or proximate the snare loop and/or the target tissue. The return electrode may be formed in the loop and/or at a tubular structure proximal of the loop.

As the current is applied to the target tissue, the target tissue may be cut 206 with the snare loop and the current applied to the active electrode along the snare loop. In some examples, a controller coupled with the active electrode may apply a current flow through the active electrode to the return electrode to excise and/or cauterize the target tissue as the snare loop engages the target tissue. Once the target tissue has been excised, the excised tissue may be removed from the body cavity and/or body lumen of the subject in any suitable manner including, but not limited to, aspiration, a basket, etc.

The schematic methods and techniques discussed herein may be used together, unless expressly indicated otherwise. Further, the order of implementation of the steps discussed herein may be in other suitable orders, unless expressly indicated otherwise, may include one or more intervening steps discussed herein or otherwise.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A medical device, comprising:
a first electrically conductive shaft comprising a first electrically insulating material defining an outer surface of the first electrically conductive shaft;
a second electrically conductive shaft; and
a loop configured to be in electrical communication with and to extend in a distal direction from the first electrically conductive shaft and the second electrically conductive shaft, a portion of the loop having an electrically conductive circumferential outer surface, and
wherein the first electrically conductive shaft, the second electrically conductive shaft, and the loop form a bipolar snare.

2. The medical device of claim 1, wherein the first electrically conductive shaft comprises an electrically conductive coil with the first electrically insulating material on an outer surface of the electrically conductive coil.

3. The medical device of claim 2, wherein the second electrically conductive shaft is configured to extend through a lumen defined by the electrically conductive coil and the second electrically conductive shaft comprises a second electrically insulating material defining an outer surface of a portion of the second electrically conductive shaft extending within the lumen.

4. The medical device of claim 1, further comprising:
an electrically conductive ring coupled with a distal end of the first electrically conductive shaft, and
wherein the electrically conductive ring is a portion of a return electrode of the bipolar snare.

5. The medical device of claim 4, wherein the electrically conductive ring defines a lumen configured to receive the second electrically conductive shaft and the loop, and wherein the lumen is defined by an inner surface of a second electrically insulating material.

6. The medical device of claim 5, wherein the first electrically conductive shaft comprises an electrically conductive coil and the second electrically insulating material extends into a lumen defined by the electrically conductive coil.

7. The medical device of claim 4, wherein the electrically conductive ring has an electrically conductive outer surface.

8. The medical device of claim 4, wherein the loop is partially insulated to prevent direct electrical contact between the loop and the electrically conductive ring.

9. The medical device of claim 1, wherein the first electrically conductive shaft and the second electrically conductive shaft define the loop.

10. The medical device of claim 9, wherein the first electrically insulating material extends along the loop and defines one or openings through which the first electrically conductive shaft is configured to receive current from a portion of the second electrically conductive shaft defining the loop.

11. The medical device of claim 1, wherein the loop comprises a plurality of wires, one or more of the plurality of wires extends from the first electrically conductive shaft and has an outer surface defined by a second insulating material defining one or more openings, and the loop has a distal portion comprising the one or more openings and a proximal portion.

12. A medical device, comprising:
a return electrode defining a lumen;
an active electrode comprising:
a control shaft; and
a loop coupled with the control shaft and configured to translate with the control shaft relative to the lumen;
wherein the return electrode is configured to receive current from the active electrode through tissue of a patient.

13. The medical device of claim 12, wherein the return electrode has a proximal region and a distal region, the proximal region has an outer surface defined by a first electrically insulating material and the distal region has an outer surface defined by an electrically conductive material.

14. The medical device of claim 13, wherein a second electrically insulating material defines an inner surface of the return electrode extending in a proximal direction from a distal end of the distal region.

15. The medical device of claim 12, further comprising:
an electrically insulating material defining an outer surface of the control shaft and the loop.

16. The medical device of claim 12, wherein the control shaft comprises one or more wires and the one or more wires define the loop.

17. A medical device, comprising:
an outer shaft defining a lumen;
a control shaft configured to extend through the lumen; and
a loop coupled with the control shaft and configured to translate with the control shaft relative to the outer shaft, the loop is formed from a plurality of wires,
wherein a first wire of the plurality of wires is configured as an active electrode having an electrically conductive circumferential outer surface defining a portion of the loop, and
wherein a second wire of the plurality of wires is configured as a return electrode configured to receive current from the active electrode through tissue of a patient, the second wire having an electrically insulating circumferential outer surface defining a portion of the loop and one or more openings in the electrically insulating circumferential outer surface configured to receive the current from the first wire.

18. The medical device of claim 17, wherein the control shaft comprises the first wire and the second wire.

19. The medical device of claim 17, wherein two or more of the plurality of wires are configured as the active electrode.

20. The medical device of claim 17, wherein the loop has a proximal portion and a distal portion, the distal portion comprising the one or more openings.
